(19) United States
(12) Patent Application Publication
(10) Pub. No.: US 2003/0176483 A1
(43) Pub. Date: Sep. 18, 2003

(54) Topical synergistic microbicide

(76) Inventor: Gerald N. Kern, Scottsdale, AZ (US)

Correspondence Address: Greenberg Traurig, LLP, 21st Floor, 885 Third Avenue, New York, NY, 10022, US

(21) Appl. No.: 10/096,672

(22) Filed: Mar. 13, 2002

(51) Int. Cl. A61K 31/4178; A61K 31/275; A61K 31/24; A61K 31/195; A61K 31/10

(52) U.S. Cl. 514/397; 514/567; 514/526; 514/536; 514/708; 514/739

(57) ABSTRACT

Application of topical synergistic microbicide composition(s) of high practical utility both as a treatment and/or prevention against HSV Type 1 and/or Type 2 and related diseases and secondary opportunist disease causing agents, while reducing the inflammation, prolonged healing times and reduce scaring.

### FIELD OF THE INVENTION

Method and pharmaceutical composition for the prevention and/or treatment of Herpes Simplex Virus and related diseases including secondary opportunist disease causing agents.

### BACKGROUND OF THE INVENTION

The present invention relates to a time and dose dependant synergistic composition and method of administration for the inactivation of Herpes Simplex Virus and related diseases.

Sexually transmitted infections (STIs), such as genital herpes, are a major public health problem worldwide. It is estimated that several hundred million individuals are infected with one or more STI (for reviews see Gerbase et al., 1998; Desormeaux et al., 1999). One desired approach for control of transmission has been the development of nontoxic, topical, broad-spectrum microbicides effective against the transmission of these pathogens. Several categories of microbicides have been developed or are currently in clinical trials. These include spermicides/microbicides, microbial adhesion inhibitors, anti-inflammatory, anti-microbial drugs, and buffering agents and products that maintain normal vaginal environment (Desormeaux et al., 1999). One class of spermicides/microbicides currently in use is surface-active agents, such as nonoxynol-9 (N-9), SDS, or benzalkonium. Such agents are known to inactivate pathogens by disrupting an organism's membrane or viral envelope, although this may not be the complete story, since SDS has also been shown to inactivate the non-enveloped virus HPV (papillomavirus) (Howell et al., 1999). However, the major problem associated with the use of such microbicides has been local high levels of irritation, inflammation and/or ulceration of mucosae. The present invention as described herein alleviates such problems of currently used microbicide compositions by the administration of time and dose dependant effective synergistic composition(s) as claimed herein.

It is known that herpes simplex virus (HSV) has a worldwide distribution and causes the most common infections affecting a mammal especially humans. Herpes simplex occurs in two antigenic types—that have a tendency to overlap—, HSV-1 generally causes herpes labialis, adult herpes encephalitis and keratoconjunctivitis, while HSV-2 causes Herpes genitalis and lower extremity herpes referred to as Type 1 and Type 2 or HSV-1 and HSV-2, respectively.

Herpes simplex virus (HSV) causes infections characterized by symptomatic inflammation of targets such as skin and mucosal membranes of the lower abdomen including the genital areas. The type of HSV most commonly associated with lesions of the mouth and facial region is Type 1 HSV. Type 2 HSV is most commonly associated with lesions of the genital region. Symptoms initially include tingling, pain, and swelling at the target site of infection, followed and characterized by mild to severe fluid-filled vesicular lesions, referred to as cold sores or fever blisters, these painful symptoms are very common even after application of currently used methods and compositions.

The points of entry of pathogenic viruses and secondary pathogenic bacteria or fungi are predominantly the skin and mucus membranes, including the upper and lower respiratory tracts. The first step in many infections is attachment or colonization on skin or mucus membranes, followed by subsequent invasion and dissemination of secondary infectious pathogens.

Followed by poor healing times and recurrent infection of secondary pathogenic infection from opportunist pathogens, the target site of the wound is more susceptible to extensive dehydration and prolonged scar formation of the surrounding area thereby impeding the normal healing process time and subjecting the patient to unacceptable prolonged levels of pain and physical discomfort.

While no complete cure is presently known for a herpes simplex and the associated secondary infections caused by opportunist disease causing agents, certain substances have been advanced for the management of the disease. In particular the administration of 3,3-bis(p-hydroxyphenyl) phthalide in amounts up to 100 milligrams (15 to 30 milligrams initially and repetitively at predetermined intervals) has shown to be ineffective. Interferon has also been tested systemically and topically against HSV but with only limited efficacy. There are many side effects associated with systemic administration of anti-virals that topical treatments are greatly preferred. The use of nonoxynol-9 (N-9), or benzalkonium have shown detrimental problems with the application of their active ingredients that inevitably cause distinct irritation to the already inflamed target site or wound area, thereby elevating the problems and instigating the onset of secondary infection from opportunist pathogens.

Topical applications used today for treatment of such related diseases as for example HSV have the recurrent problem of containing skin irritants within topical formulations used for carriers and/or active ingredients that not only prolong the painful complications including increased: inflammation and irritation, but also impede healing times concurrent with poor inadvertent results i.e. compromised sites open to further infection from opportunistic disease causing agents. As a result the suffering continues to unsatisfactory levels leaving the patient with further unnecessary problems both physical and emotional.

Detergents and solvents have been used to inactivate viruses in blood or plasma derived products, as described in U.S. Pat. No. 4,540,573 to Neurath, the contents of which are hereby incorporated by reference. This patent teaches the use of aqueous solutions of trialkylphosphates to inactivate viruses in blood-derived products in vitro.

However, after treatment of the blood derived product according to this method, the trialkyl phosphate must then be removed by methods such as dialysis or lyophilization, necessitating additional extraction steps (as described in U.S. Pat. No. 4,909,940 to Horowitz, the contents of which are hereby incorporated by reference). This lengthy and complex procedure is not suitable for use in treating viral infections in a affected mammal, neither does the patent teach or suggest in vivo uses of the solutions.

Furthermore, many trialkyl phosphates are known to be toxic or irritating to mucous membranes (see, e.g., Smyth and Carpenter (1944) J Ind. Hyg. Toxicol. 26: 269). For these reasons, they have not been thought to be usable in preparations intended for topical in vivo administration.

Within the three known product applications employed for percutaneous drug absorption via the skin barrier i.e. cosmetic, topical and transdermal applications for conditions, the optimal delivery strategy for administration of pharmaceuticals via the skin varies based on individual pharmaceuticals and among varying diseases and their disease states.

Accordingly, it is desirable to provide a local topical synergistic composition suited for the condition to be treated or prevented for the skin which on route—via the appropriate carrier(s) to—inactivates the disease causing agents including herpes simplex virus, cytomegalovirus, Epstein-Barr virus, varicella zoster virus, influenza virus, human lymphotrophic virus, human immunodeficiency virus, papilloma virus or respiratory syncytial virus and other related pathogens.

Therefore there is a need in the art for improved methods and compositions for the prevention and treatment of disease causing agents including specifically HSV Type 1 and Type 2, cytomegalovirus, Epstein-Barr virus, varicella zoster virus, influenza virus, human lymphotrophic virus, papilloma virus or respiratory syncytial virus and secondary opportunist pathogenic micro-organisms accompanied with decreased inflammation, pain relief and reduced healing times.

### SUMMARY OF THE INVENTION

By this invention there is provided a pharmaceutical acceptable microbicide of active composition(s) selected from at least one preservative in combination with one or more analgesic(s) and keratolytic agent(s) for an unexpected effective treatment and/or prevention of herpes viruses and related diseases including but not limited to cytomegalovirus, Epstein-Barr virus, varicella zoster virus, influenza virus, human lymphotrophic virus, papilloma virus or respiratory syncytial virus.

More specifically this invention involves the application of microbicides in a dose and time dependant synergistic composition for inactivating or preventing HSV Type 1 and Type 2, varicella zoster virus, human lymphotrophic virus, papilloma virus or respiratory syncytial virus.

The aforementioned methods for treating or preventing infections from primary and secondary opportunist pathogens include such compositions and methods as using and applying skin patches and topical lotions, creams, pastes, balms, gels, salves and ointments, respectively, as carrier(s) for the active synergistic composition(s) as defined herein.

The synergistic microbicide compositions of the present invention includes but are not limited to time and dose dependant pharmaceutical acceptable preservative(s) such as dioctyl-sulfosuccinate and salts or mixtures thereof combined with one or more keratolytic agents selected from the following group, including, algestone, acetophenide, p-aminobenzoic acid and salts thereof, cyoctol, glyoxyl diureide, metronidazole, and analgesics including lidocaine, benzocaine and 2,2′,2″-Nitrilotriethanol or mixtures thereof.

The microbicide composition of the present invention can also include in predefined dose and time dependant synergistic quantities the following anti-inflammatory agent(s) selected from the group consisting of 4,4′-diaminodiphenyl sulfone and behenyl alcohol or mixtures thereof.

The surprising efficacious activity of such time and dose dependant synergistic composition(s) of the present invention has shown to have high practical utility both as a treatment and prevention against HSV type 1 or type 2 and related diseases and against secondary opportunist disease causing agents.

### DETAILED DESCRIPTION

Accordingly, an object of the present invention is to provide virus-inactivating synergistic time and dose dependant composition(s) that has shown to inactivate viruses and related diseases on contact with a mammal in need thereof.

It is a further object of the invention to provide a non-irritating, non-inflammatory, stable pharmaceutical acceptable preparation that inactivates viruses, especially envelope and non-envelope viruses, on the skin or mucous membranes and to provide a deterrent for both primary and secondary pathogens as described herein.

Another object of the present invention is to provide a method of inactivating a virus by contacting the virus with a virus-inactivating synergistic composition. A further object of the present invention is to provide a method of preventing and/or treating a viral infection in an affected mammal's by administering a virus-inactivating time and dose dependant synergistic composition, of the present invention, to a human in need thereof.

An even further object of the present invention is to provide a method of preventing or treating herpes simplex virus (HSV) type 1 and type 2 in a dose and time dependant fashion of a mammal in need thereof.

An ever further object of the present invention is to provide a method of alleviating the primary and secondary problems associated with HSV and to provide a mammal with reduced healing times.

It is a further object of the present invention to provide topical time and dose dependant composition(s) for treatment or respective prevention of the symptoms of HSV Type 1 and Type 2 infection and related secondary infections with minimal if not negligible side effects and relief from pain, inflammation, prolonged healing times and reduced tissue scaring as a result of viral and other secondary microbial induced lesions for a mammal in particular a human.

It is a further object of the present invention to prevent the spread of opportunist pathogens including but not limited to HSV types 1 and HSV Types 2 to surrounding susceptible warm-blooded mammalian epithelial cells.

In a further preferred embodiment of the present invention the method and composition(s) provided for preventing or treating such microbial pathogenic infections would incorporate the use of one or more compounds or family of time and dose dependant composition(s) having synergistic effects that can be topically administered to a mammal, thereby preventing a plurality of viral and other opportunist microbial infections from prolonging the associated irritation, inflammation, pain and misery.

In the methods of the present invention, the synergistic combination of preservative(s), analgesic(s), keratolytic agent(s) and anti-inflammatory agent(s) or mixtures thereof, herein described in detail, are typically administered in admixture with suitable pharmaceutical carriers (collectively referred to herein as “carrier” materials) suitably selected with the respect to the intended form of administration, that is, skin patches, sprays, gels, balms, creams, powders and the like, and consistent with conventional pharmaceutical practices.

In an embodiment of the present invention there is disclosed topical pharmaceutical composition(s) in the form of a solution, cream, ointment, gel, lotion, shampoo, aerosol formulation or skin patches adapted for application to the skin.

In a preferred embodiment of the present invention there is provided topical pharmaceutical composition(s) for the treatment and/or prevention of HSV and related diseases in dose dependant composition comprising from about 0.001% to 15% by weight each of preservative(s), keratolytic agent(s) and analgesic(s) in admixture with an anti-inflammatory agent(s) and a pharmaceutical acceptable carrier.

In a further preferred embodiment of the present invention there is disclosed topical pharmaceutical composition(s) for the treatment and/or prevention of HSV and related diseases in time dependant topical administration(s) including applying the composition—depending on the condition of the severity of symptoms—at least once a day, preferably late at night for5 days.

Topical preparations containing the combination active drug component(s) can be admixed with a variety of carrier materials well known in the art, such as, e.g., alcohols, aloe vera gel, glycerin, Vitamin A and E oils, mineral oil, and the like, to form, e.g., alcoholic solutions including behenyl alcohol, topical cleansers, creams, skin gels, skin lotions in cream or gel formulations.

The following examples are provided to further illustrate the composition(s) and method of using synergistic combinations for the effective prevention and treatment of HSV Type 1 and Type 2 and related diseases. The examples are not intended to be limitations on the scope of the instant invention in any way, and they should not be construed as such. Furthermore, the compounds described in the following examples are not to be construed as forming the only genus that is considered as the invention, and any combination of compounds or their moieties may itself form a genus.

### EXAMPLE 1

Dose and Time Dependent Synergistic Composition(s)

(0.001% to 15% by weight)

Preservative(s):

1,2-dinonyl ethyl sodium sulfate and

dioctyl-sulfosuccinate and salts or mixtures thereof.

Virus and Cells

HSV strains G (HSV-2) and F (HSV-1) were used in the experiments. A HSV susceptible cell line, Vero cells (African green monkey kidney cell), was used in the virus yield reduction assays. The culture medium for Vero cells was 5% minimum essential medium (MEM; GIBCO/BRL) supplemented with 5% fetal bovine serum, 100 U/ml penicillin and 100 μg/ml streptomycin).

Virus Plaque Reduction Assay

Antiviral effects of selected preservative(s) were determined by modified plaque reduction assays. Confluent cells were washed with PBS and subsequently infected with HSV for 1 h at 37° C. After viral inoculum was removed, the infected cells were washed with PBS and overlaid with 0.5% methylcellulose in culture medium. Cells were incubated at 37° C. for 2 days for HSV-2 infection and 3 days for HSV-1. When plaque size was adequate, cells were fixed with 10% formalin for 10 mm. Monolayers were subsequently stained with 0.5% crystal violet for 10 min, and dye was removed by washing with tap water.

All data were generated from duplicate or triplicate experiments. Mean plaque counts are shown in the Tables and Figures. Effects of compounds at varying concentrations were expressed as % of control (the mean plaque counts in drug treated wells/the mean plaque counts in control wells).

Inactivation of HSV by Selected Preservative(s)

HSV G and F strains were diluted to 200 pfu/ml with 5% MEM, respectively. The selected preservative(s) and SDS were diluted to 2×final concentrations (final concentrations: 0, 0.0005%, 0.001%, 0.0025%, 0.005% and 0.01%, respectively) with 5% MEM. Equal volumes of diluted virus and drugs were mixed and incubated at 37° C. water-bath for 1 h. One ml of the mixture was then used to infect confluent Vero cells in 6-well plates at 37° C. for 1 h. After infection, viral inoculum was removed and the cells washed with PBS. The cells were subsequently overlaid with 1.5 ml of 0.5% methylcellullose (diluted from 1% stock with 2×culture medium) for plaque assay.

To examine the kinetics of inactivation of HSV by the preservative(s), 200 pfu/ml of HSV-2 was pre-mixed with 2×final concentrations of preservative(s) (final concentrations:0, 0.001%, 0.0025%, 0.005% and 0.01%, respectively) in 5% MEM and incubated at 37° C. for 0, 15, 30, 60, 120, and 240 mm. At each time point, the treated mixture was used to infect confluent Vero cells. After viral inoculum was removed, the cells were covered with methylcellullose for plaque assay.

Cytotoxicity of the Preservative(s)

The cytotoxicity of preservative(s) was examined using Vero cells with the neutral red uptake assay described by Schmidt and Korba (2000). Culture medium was removed from confluent Vero cells in 24-well plates. The cells were then washed once with PBS. One ml of culture medium containing the preservative(s) or SDS at concentrations of0, 0.001%, 0.0025%, 0.005%, and 0.01% were added to each well. Cells were incubated at 37° C. for 1 h, 6 h, 2 and 3 days. At each time point, the medium was removed and cells washed with PBS. 500 μl of 0.01% neutral red (in PBS) was added to each well, and the samples were incubated at 37° C. for 30 min. The dye was then removed and the cells washed×2 with 1 ml PBS per well. The dye was extracted by addition of 500 μl of 50% ethanol/1% glacial acetic acid in PBS to each well and incubated at room temperature for 15 min with gentle shaking at 120-150 rpm. 200 μl extracted dye from each well was put into 96-well plate and the absorbance at 550 nm was read on an ELISA reader.

Pre-Treatment of Cells

Culture medium was removed from confluent Vero cells in 6-well plates and the cells were washed with PBS. One ml culture medium containing either the selected preservative(s)(s) or SDS at concentration of 0, 0.0005%, 0.001%, 0.0025%, 0.005% and 0.01% was added to each well and the cells were incubated at 37° C. for 1 h. Following pre-incubation of the cells with preservative(s), two treatments were performed: one set of plates was directly infected with 100 pfu/well of HSV-2; another set of plates was washed 3 times with PBS, and then infected with 100 pfu/well of HSV-2. Both infections were incubated at 37° C. for 1 h with tilting every 10 min. After viral inoculum was removed, the infected cells were covered with 1.5 ml of 0.5% methylcellullose in culture medium, and incubated at 37° C. for 2 days until the monolayers were stained and plaques counted.

Effect of Preservative(s) on HSV-Infected Cells

Confluent Vero cells were washed with PBS and then infected with 100 pfu/well of HSV-2 at 37° C. for 1 h. Following removal of viral inoculum, infected cells were washed once with PBS and covered with 0.5% methylcellullose containing either preservative(s) or SDS at concentrations of 0, 0.0005%, 0.001%, 0.0025%, 0.005%, and 0.01%. The cells were incubated at 37° C. for 2 days for plaque assay.

Effect of Preservative(s) on HSV Drug-Resistant Strains

The antiviral effect of preservative(s) on HSV-2 thymidine kinase mutant Delta 333 and HSV-1 DNA polymerase mutant 615.8 was performed in a similar way as above (see section 2.4)

### Results

Inactivation of HSV

The in vitro inactivations of HSV by selected preservative(s) were compared with the SDS. Various concentrations of testing drug were pre-mixed with HSV-1 or HSV-2 and incubated at 37° C. for 1 h. Antiviral effects were then determined by plaque reduction assays (FIG. 1). Inactivation of HSV-1 by preservative(s) along with SDS is shown in FIG. 1A and that of HSV-2 in FIG. 1B. Results showed that at a concentration of 0.005% preservative(s) completely inactivated both HSV-1 and HSV-2 after pre-mixing for 1 h (EC₉₀₋₁₀₀=0.005%). In contrast, although this concentration was also effective for SDS, it caused 50% cell death (toxic to cells). At concentration of 0.0 1%, both preservative(s) and SDS were shown to be toxic to the cells causing 50-100% cell death (data not shown). In these experiments, the selected preservative(s) showed to be more effective against HSV-1 than HSV-2, and slightly more potent and less toxic than SDS at other concentrations (FIG. 1).

The Kinetics of Inactivation of HSV by Preservative(s)

To examine the kinetics of inactivation of HSV, the preservative(s) at various concentrations was mixed with HSV-2 and incubated at 37° C. in a time-course, with endpoints determined by plaque reduction assay. Results (FIG. 2) showed that the preservative(s) had a slight inactivation effect upon HSV-2 at concentrations of 0.001% and 0.0025% after 4 h incubation. However, inactivation reached 50% at time 0, almost 90% (EC₉₀) after 30 mm, and 100% after I h incubation at a concentration of 0.005%. A concentration of 0.01% of preservative(s) was found to be toxic to cells.

Short and Long Incubation Cytotoxicities

Microbicidal effectiveness of surface-active agents such as preservative(s) against enveloped viruses suggests a potentially disruptive effect on cellular membranes. Accordingly, experiments were carried out to evaluate relative cytotoxicities of preservative(s) over time. Cytotoxicities of preservative(s) were compared with SDS or sarcosine and measured using Vero cells. Trypan blue exclusion (data not shown) and uptake of neutral red dye were then used to determine the viabilities of cells after incubations with different concentrations of preservative(s), SDS, and sarcosine. Results were consistent between assays (data not shown). For short-term incubation, cells were exposed to preservative(s) for 1 h (FIG. 3A) and 6 h (data not shown), whereas for long-term incubation, cells were exposed to preservative(s) for 2 (data not shown) and 3 days (FIG. 3B). As can be seen from these figures, after 1 h exposure to preservative(s) and SDS, minimal cytotoxicity of preservative(s) to Vero cells was observed even at concentration of 0.01%. Cytotoxicity was increased after 6 h exposure (data not shown). Concentration at 0.01% was again found to be toxic to Vero cells after 3 days' incubation. Thus, the cytotoxicity of preservative(s) is time and dependent. Furthermore, preservative(s) was slightly less cytotoxic than either SDS or sarcosine (data not shown for sarcosine, similar cytotoxicity was observed for both SDS and sarcosine). CC₅₀ (cytotoxic concentration giving 50% of cell death) of preservative(s) after 2 days' incubation was approximately 0.01% and that of SDS and sarcosine was approximately 0.005%, respectively (data not shown).

Antiviral Activity of Preservative(s) Determined by Pre-Treated Cells

Some compounds can be internalized into cells or bound to the cellular membranes to exert antiviral effects. This experiment was designed to examine the effect of preservative(s) on pre-treated cells. Two approaches were employed. First, we preincubated Vero cells with preservative(s) and then infected these cells with HSV. In the second, we pre-incubated cells with preservative(s) and then washed it off with PBS×3. The results, together with comparison of SDS, were summarized in Table 1. Cells pretreated with preservative(s) at a concentration of 0.005%, showed virus infection to be reduced by 45%, and 35% after preservative(s) was removed by three washes. However, this concentration was not able to completely stop virus infection.

Effect of Preservative(s) on HSV-Infected Cells

To examine the effect of preservative(s) on HSV-infected cells, cells were first infected with HSV-2 and then treated with different concentrations of preservative(s) following adsorption by inclusion in the methylcellullose overlay. The results (Table 2) indicated that preservative(s) only had slight inhibitory effect on HSV-infected cells, by approximately 30% plaque reduction at 0.005% of concentration.

Effect of Preservative(s) on HSV-Drug-Resistant Mutants

Delta 333 is a laboratory HSV-2 TK⁻ (thymidine kinase deleted) mutant strain with genotypic and phenotypic resistance to both penciclovir and acyclovir. To test the antiviral effect of preservative(s) on this drug-resistant HSV strain, delta 333 virus was pre-mixed with different doses of preservative(s) at 37° C. for 1 h. The antiviral effect was subsequently determined by plaque reduction assay. The results showed that preservative(s) at concentration of 0.005% could completely inactivate delta 333 after incubation at 37° C. for 1 h (data not shown). Although SDS was slightly less efficacious in this assay, it was also capable of inactivating the resistant strain, delta 333.

HSV-1 drug-resistant strain 615.8 is a DNA polymerase mutant with phenotypic resistant to forscarnet (Sacks et al., 1989). The effect of preservative(s) on the polymerase mutant virus was examined by pre-mixing preservative(s) at various doses with 615.8 virus for 1 h at 37° C. The antiviral activity against polymerase mutant virus was then determined by plaque reduction assay. The results showed that preservative(s) at concentration of 0.005% could inactivate approximately 93% of polymerase mutant 615.8 after incubation at 37° C. for 1 h (data not shown).

The following are non-limiting examples of uses of synergistic microbicide compositions.

### EXAMPLE 2

Topical Cream Administration

An exemplary embodiment of time and dose dependant creams prepared for topical administration for treating and preventing viral infection in accordance with practice of principles of this invention comprises 0.2% wt/wt preservative(s), and about 5.0 to about 20% by weight of analgesic(s) and about 0.01 to about 3.0% by weight of keratolytic agent(s) and from about 0.01 to about 5.0% of anti-inflammatory agent(s). 5% wt/wt mineral oil, 4.5% wt/wt stearic acid, 3.5% wt/wt cetyl alcohol, 1.5% wt/wt triethanolamine, 0.15% wt/wt methylparaben, 0.05% wt/wt with the remainder being deionized water.

Local application times are measured in accordance with the dose and severity of conditions of the mammal's. The times of application range from about 1 to 12 hours once or twice a day, one administration every 2 to 3 days with increased wt/wt preservative of 3.0 to about 6.0%. Though these times and dose regimes have shown to be flexible and are applied as such in accordance with the state of the mammal in need thereof.

Accordingly the cream is prepared in two parts microparticulate A which includes mineral oil, stearic acid, cetyl alcohol, and microparticulate B which includes deionized water, triethanolamine, and methylparaben. To prepare the cream, an appropriate amount of mineral oil is metered into a jacketed stainless steel vessel. Into the same vessel, an appropriate amount of each of stearic acid, cetyl alcohol and methylparaben is measured.

Propeller agitation is provided and microparticulate A—oil microparticulate—is heated from about 50.degree. to 60.degree. C. Mixing is continued until all micro-particulates are melted and a clear cream is obtained. Microparticulate B is then obtained by weighing an appropriate amount of water into a jacketed stainless steel vessel, which is provided with a sweep agitation. Into the same vessel, an appropriate amount of each of preservative(s), keratolytic agent(s) and analgesic(s) is added. Additionally at this stage of the process it is optional to include the anti-inflammatory agent(s). Microparticulate B is brought to 60.degree. to 65.degree. C. with gentle propeller agitation to obtain a clear cream. Microparticulate A is then added to microparticulate B which from about 60.degree.70.degree. C. with continued propeller agitation. Mixing is continued for 40 minutes with the combined microparticulates from about 65.degree. To about 75.degree C. The batch is then cooled by introducing cooling water into the jacket of the vessel. Cooling is continued with moderate propeller agitation. When the batch begins to thicken agitation is continued until batch temperature reaches from about 25.degree. to about 40.degree. C. A sample is then taken from both the top and bottom of the batch for quality control analysis.

The product is then ready to be placed into containers for topical application. As an example a person who has HSV Type 1 or Type 2 infection with active facial or genital lesions applies the cream to the afflicted site periodically. In one embodiment the cream is applied every 8 hours. In another embodiment the cream is applied twice daily every 4 hours. The treatment is continued until the afflicted area(s) are healed.

While the above examples illustrate the use of one or more exemplary cream formulation(s), alternative time and dose related composition(s) are contemplated which include pharmaceutically acceptable carrier(s) other than those of this embodiment.

Additionally, the percentage of active synergistic composition used can be different in accordance with the mammal's condition.

For example, cream formulations having as little as 0.001% to greater than 4.0% preservative(s), 4.0% of anti-inflammatory agent(s)—if and when required—, 2.0% analgesic(s) and 5.0% by weight of keratolytic agent(s) of a mammal with severe conditions, compromised immune system and of fair skin are contemplated.

What is claimed is:

1. A pharmaceutical composition useful for treating viral infections in humans and animals which comprises a therapeutically effective amount of at least one preservative or pharmaceutically acceptable salt thereof in combination with a synergistically effective amount of at least one keratolytic agent or a pharmaceutically acceptable salt thereof, in combination with a pharmaceutically acceptable carrier.

2. A composition according to claim 1 which additionally comprises an effective amount of an analgesic.

3. A composition according to claim 2 which additionally comprises an effective amount of an anti-inflammatory agent.

4. A composition according to claim 1 wherein the preservative is a straight or branched chain alkyl moiety of 5 to 12 carbon atoms.

5. A composition according to claim 4 wherein the preservative is selected from the group consisting of 1,2-dinonylethyl sodium sulfonate and salts thereof, dioctyl-sulfosuccinate and salts thereof, and mixtures of 1,2-dinonylethyl sodium sulfonate and salts thereof in combination with dioctyl-sulfosuccinate and salts thereof.

6. A composition according to claim 5 wherein the preservative is dioctyl-sodium sulfosuccinate.

7. A composition according to claim 6 wherein the dioctyl-sodium sulfosuccinate is present in a concentration of 0.25%.

8. A composition according to claim 5 wherein the preservative is in the form of the calcium, sodium, potassium or magnesium salt.

9. A composition according to claim 8 wherein the salt is calcium.

10. A composition according to claim 8 wherein the salt is sodium.

11. A composition according to claim 1 wherein the keratolytic agent is selected from the group consisting of p-aminobenzoic acid and salts thereof, glyoxyl diureide, cyoctol, metronidazole, and mixtures thereof.

12. A composition according to claim 11 wherein the p-aminobenzoic acid salt is potassium.

13. A composition according to claim 2 wherein the analgesic is lidocaine, benzocaine, 2,2′, 2″-nitrilotriethanol, or mixtures thereof.

14. A composition according to claim 3 wherein the anti-inflammatory agent is 4,4′-diaminodiphenyl sulfone, behenyl alcohol or mixtures thereof.

15. A composition according to claim 3 in topical application forms.

16. A composition according to claim 15 wherein the topical forms consist of skin patches, aerosol formulation, shampoo, lotions, creams, balms, gels, salves and ointments.

17. A composition according to claim 15 wherein the preservative, the keratolytic agent, the analgesic and the inflammatory agent are all present in synergistic proportions.

18. A composition according to claim 17 wherein the preservative, the keratolytic agent and the analgesic are present in a ratio of 3:2:1.

19. A composition according to claim 18 wherein the preservative, the keratolytic agent and the analgesic are present in an amount of between 0.001 to 15% by weight.

20. A composition according to claim 17 which comprises about 0.001 to about5% by weight of preservative or pharmaceutically acceptable salt thereof, about 0.01% to about 5.0% by weight of keratolytic agent or pharmaceutically acceptable salt thereof and about 5.0 to about 20% by weight of analgesic.

21. A composition according to claim 20 wherein the amounts by weight are about 0.2% by weight of preservative or pharmaceutically acceptable salt thereof, about 0.01% to about 5.0% by weight of keratolytic agent or pharmaceutically acceptable salt thereof, about 5.0 to about 20% by weight of analgesic, and from about 0.01% to about 5.0% of anti-inflammatory agent.

22. A method of treating or preventing a human or animal afflicted by or susceptible to Herpes Simplex Virus Type 1 and Type 2 and related diseases comprising topically administering a pharmaceutical composition which comprises of a therapeutically effective amount of at least one preservative in combination with a pharmaceutically acceptable carrier.

23. A method according to claim 22 wherein the composition further comprises a synergistically effective amount of a keratolytic agent or pharmaceutically acceptable salt thereof.

24. A method according to claim 23 wherein the pharmaceutical compositions further comprises of an effective amount of an analgesic compound.

25. A method according to claim 24 wherein the pharmaceutical compositions further comprises an effective amount of an anti-inflammatory.

26. A method according to claim 22 wherein the preservative is a straight or branched chain alkyl moiety of 5 to 12 carbon atoms.

27. A method according to claim 26 wherein the preservative is selected from the group consisting of 1,2-dinonylethyl sodium sulfonate and salts thereof, dioctyl-sulfosuccinate and salts thereof, and mixtures of 1,2-dinonylethyl sodium sulfonate and salts thereof in combination with dioctyl-sulfosuccinate and salts thereof.

28. A method according to claim 27 wherein the preservative is dioctyl-sodium sulfosuccinate.

29. A method according to claim 28 wherein the dioctyl-sodium sulfosuccinate is present in a concentration of 0.25%.

30. A method according to claim 27 wherein the preservative is in the form of the calcium, sodium, potassium or magnesium salt.

31. A method according to claim 30 wherein the salt is calcium.

32. A method according to claim 30 wherein the salt is sodium.

33. A method according to claim 23 wherein the keratolytic agent is selected from the group consisting of p-aminobenzoic acid and salts thereof, glyoxyl diureide, cyoctol, metronidazole, and mixtures thereof.

34. A method according to claim 33 wherein the p-aminobenzoic acid salt is potassium.

35. A method according to claim 24 wherein the analgesic compound is lidocaine, benzocaine, 2,2′, 2″-nitrilotriethanol, or mixtures thereof.

36. A method according to claim 25 wherein the anti-inflammatory compound is 4,4′-diaminodiphenyl sulfone, behenyl alcohol or mixtures thereof.

37. A method according to claim 25 wherein the preservative, the keratolytic agent, the analgesic and the inflammatory agent are all present in synergistic proportions.

38. A method according to claim 24 wherein the preservative, the keratolytic agent and the analgesic are present in a ratio of 3:2:1.

39. A method according to claim 38 wherein the preservative, the keratolytic agent and the analgesic are present in an amount of between 0.001 to 15% by weight.

40. A method according to claim 22 wherein the composition comprises about 0.001 to about 5% by weight of preservative or pharmaceutically acceptable salt thereof, about 0.01% to about 5.0% by weight of keratolytic agent or pharmaceutically acceptable salt thereof and about 5.0 to about 20% by weight of analgesic.

41. A method according to claim 40 wherein the composition comprise amounts about 0.2% by weight of preservative or pharmaceutically acceptable salt thereof, about 0.01% to about 5.0% by weight of keratolytic agent or pharmaceutically acceptable salt thereof, about 5.0 to about 20% by weight of analgesic, and from about 0.01% to about 5.0% of anti-inflammatory agent.

42. A method of treating and/or preventing cold sores and lesions associated with genital and facial HSV Type 1 and Type 2 and related diseases in humans and animals which comprises topically administering the composition of claim 1.

43. A method of preventing associated secondary infections of HSV Type 1 and Type 2 and related diseases in humans and animals, which comprises topically administering the composition of claim 1.

44. A method of ameliorating healing of painful cold sores and lesions associated with genital and facial HSV Type 1 and Type 2 and related diseases in humans and animals, which comprises topically administering the composition of claim 1.

45. A method according to claim 22 wherein the HSV related diseases is cytomegalovirus, Epstein-Barr virus, varicella zoster virus, influenza virus, human lymphotrophic virus, papilloma virus or respiratory syncytial virus.

46. A method of claim 22 wherein the composition is administered topically from about 1 to 12 hours once or twice a day.

47. A method of claim 22 wherein the composition is administered topically from about 1 to 6 hours once or twice a day.

48. A method of claim 22 wherein the composition contains from about 3.0% to about 6% preservative and is topically administered every 2 to 3 days.
